(19) United States
(12) Patent Application Publication
(10) Pub. No.: US 2017/0238851 A1
(43) Pub. Date: Aug. 24, 2017

(54) Differential Biosensor System

(71) Applicant: Echo Therapeutics, Inc., Iselin, NJ (US)

(72) Inventors: Eric V. Duhamel, Boxborough, MA (US); Kenneth Gary, Boxborough, MA (US)

(21) Appl. No.: 15/506,358

(22) PCT Filed: Aug. 26, 2015
(86) PCT No.: PCT/US2015/046982

§ 371 Date: Feb. 24, 2017

Related U.S. Application Data
(60) Provisional application No. 62/041,692, filed on Aug. 26, 2014.

(51) Int. Cl. A61B 5/1486; A61B 5/145

(52) U.S. Cl. CPC A61B 5/1486; A61B 5/14532; A61B 5/14546

(57) ABSTRACT

Sensor systems, transdermal analyte monitoring systems (TAMS), and methods of improving analyte detection are described herein. The sensor systems have two working terminals, at least one counter terminal, and optionally one or more reference terminals. The first working terminal measures electrical signals due to an analyte level along with background interferences. The second working terminal measures only background interferences. The transdermal analyte monitoring systems include the sensor system and a computing device. The computing device performs mathematical analysis using an appropriate algorithm on the electrical information provided by the electrodes of the sensor system to obtain an accurate analyte level. Typically, the second electrical signal is subtracted from the first electrical signal to obtain an accurate analyte level in real time.

### CROSS-REFERENCE TO RELATED APPLICATION

This application claims priority to provisional application, U.S. Application No. 62/041,692, filed Aug. 26, 2014, the disclosure of which is incorporated herein by reference in its entirety.

### FIELD OF THE INVENTION

The present invention is directed to the field of analyte measurement, particularly transdermal analyte measurement.

### BACKGROUND OF THE INVENTION

Diabetes is a group of metabolic disorders with one common manifestation: hyperglycemia. Persistent hyperglycemia is known to be responsible for damage to the eyes, kidneys, nerves, heart and blood vessels. The impact that diabetes has on the health of Americans is staggering. According to the American Diabetes Association in 2011, approximately 18.8 million Americans were diagnosed with diabetes. The cost of diagnosed diabetes in 2007 was estimated at $174 billion. The number of deaths in 2007 attributed to complications associated with diabetes was estimated at over 230,000 Americans.

Blood glucose monitoring is an integral part of standard diabetes care. Conventionally, a daily regimen of pricked fingers and blood tests forms an essential part of life for someone living with diabetes. Due to the lack of comfort and convenience, a diabetic will normally only measures his or her glucose level two to four times per day. Unfortunately, these time intervals are so far spread apart that the diabetic will likely find out too late, sometimes incurring dangerous side effects, of a hyper- or hypo-glycemic condition.

Analytical biosensors provide one type of system that can be used to manage diabetes. The biosensors are small and convenient, and only sample tiny amounts of fluids that lie just below the skin, making measuring glucose levels pain-free and non-invasive. Such devices combine the advantages of electrochemical signal transduction with the specificity inherent in biological interactions.

Despite recent improvements in analytical biosensor systems, the available systems suffer from disadvantages. For example, a biosensor system typically contains only one working terminal and at least one reference terminal, which are used to measure a signal associated with a concentration of the analyte in the patient. The output signal, typically a raw data stream, also includes non-analyte signals due to background interferences, such as noise due to mechanical, biochemical, and/or chemical factors. These interferences cause inaccuracies in analyte sensing. In the case of glucose sensing, these inaccuracies can cause a patient to think his/her blood glucose level is fine when it is really too high or too low. The results obtained from such devices may also cause a patient to administer a higher or lower level of insulin than needed.

New and improved systems and methods for treating and detecting diabetes are in demand.

Therefore, it is an object of the invention to provide a sensor system for improving the accuracy of detection and/or quantification of an analyte.

It is a further object of the invention to provide an improved transdermal analyte monitoring system.

It is yet another object of the invention to provide methods for improving the accuracy of detection and/or quantification of an analyte by a transdermal analyte monitoring system.

### SUMMARY OF THE INVENTION

Sensor systems, transdermal analyte monitoring systems (TAMS), and methods of improving analyte detection are described herein. The Differential Biosensor System measures in real-time all background interferences in the system and subtracts them from the analyte signal. The sensor systems have two working terminals, at least one counter terminal, and optionally one or more reference terminals. The first working terminal measures electrical signals due to an analyte level along with background interferences. The second working terminal measures only background interferences.

The transdermal analyte monitoring systems include the sensor system and a computing device. The computing device performs mathematical analysis using an appropriate algorithm on the electrical information provided by the electrodes of the sensor system to obtain an accurate analyte level. Typically, the second electrical signal is subtracted from the first electrical signal to obtain an accurate analyte level in real time. Kits for use with the transdermal analyte monitoring system TAMS are also described. And, a method for improving analyte detection using the sensor systems described herein is provided.

In a preferred embodiment, for glucose sensing, one working terminal includes the enzyme Glucose Oxidase (GOX) to convert glucose to hydrogen peroxide and thus to a current, and the other working terminal contains either a blank carbon terminal or a second enzyme, preferably as catalase, which blocks hydrogen peroxide and allows the second terminal to only measure background interferences. Using a blank carbon sensor may achieve the same effects as having an active sensor with an enzyme such as catalase. The catalase or blank carbon terminal current is then subtracted from the GOX signal, in real time either using electronics with the appropriate circuitry or in the software yielding a pure glucose signal.

### BRIEF DESCRIPTION OF THE DRAWINGS

FIG. 1 shows an exemplary analyte sensor (100), containing three terminals, where two are working terminals (102 and 104) and one is a counter terminal (106). The terminals contain leads that permit electrical connection to external devices or components.

FIG. 2 shows an exemplary 5-terminal analyte sensor (200) containing two working terminals (202 and 204), two counter terminals (206 and 208), and a reference terminal (210).

FIG. 3 shows an exemplary 6-terminal analyte sensor (300) containing two working terminals (302 and 304), two counter terminals (306 and 308), and two reference terminals (310 and 312).

FIG. 4 is a flowchart depicting an exemplary method of operation for a transdermal analyte monitoring system.

FIGS. 5A and 5B are line graphs, showing the change in various parameters measured over time with a non-glucose oxidase sensor at a first site (site 1). FIG. 5A shows the change in current (nA, black solid line) and glucose level (mg/dL, black broken line) over time (hours). FIG. 5B shows the change in current (nA, black solid line) and temperature (° C., black broken line) over time.

FIGS. 6A and 6B are line graphs, showing the change in various parameters measured over time with a glucose oxidase sensor at a second site (site 2). FIG. 6A shows the change in current (nA, black solid line) and glucose level (mg/dL, black broken line) over time (hours). FIG. 6B shows the change in current (nA, black solid line) and temperature (° C., black broken line) over time.

FIGS. 7A and 7B are line graphs, comparing the data obtained from site 2 and the data obtained from site 1 for the parameters in FIGS. 5 and 6. FIG. 7A shows the difference in current between site 2 and site 1 (nA, black solid line) and the change in glucose level (mg/dL, black broken line) over time (hours). FIG. 7B shows the change in current at site 1 (nA, black solid line) and site 2 (nA, black broken line), as well as the change in glucose level (mg/dL, black dotted line) over time (hours).

### DETAILED DESCRIPTION OF THE INVENTION

The analyte sensor system, monitoring system, kits, and methods described herein provide a means for accurately determining analyte concentration in real time in a patient. These systems, kits, and methods simultaneously measure the concentration of an analyte of interest and any non-analyte interferences which are subsequently subtracted to continuously determine an accurate analyte level.

### I. Sensor System

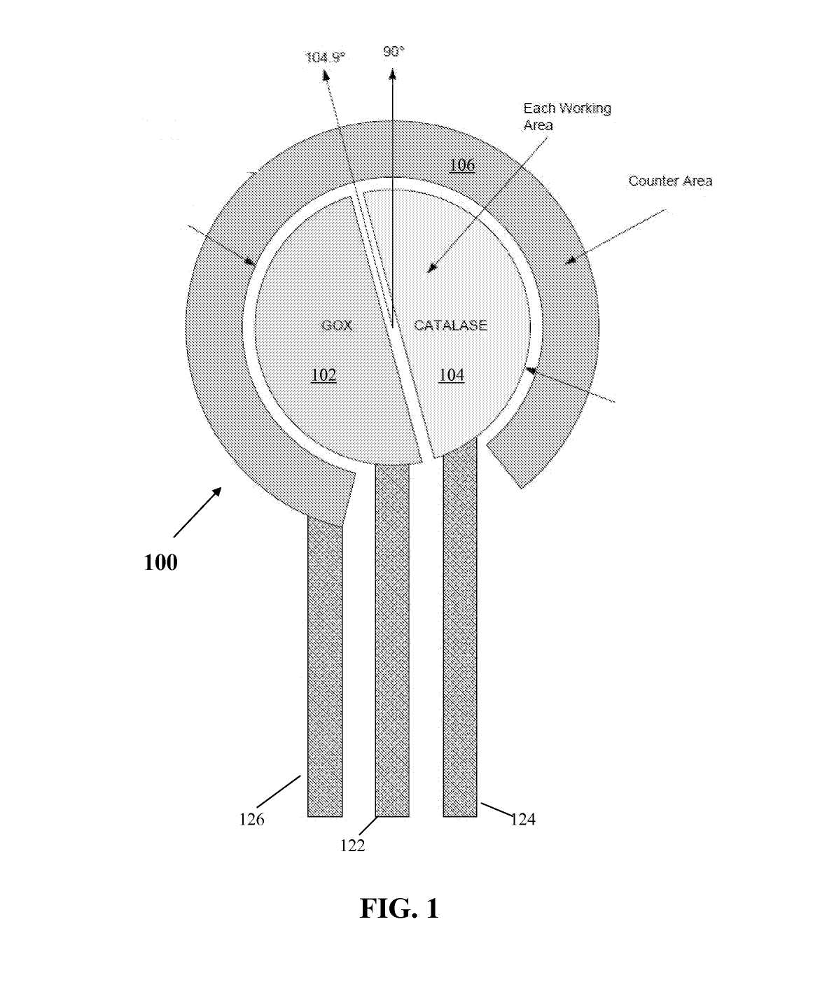

Exemplary sensor systems are illustrated in FIGS. 1, 2, and 3. The sensor systems described herein provide an analyte sensor that can discreetly or continuously measure the concentration of an analyte of interest or a substance indicative of the concentration or presence of the analyte in a body fluid. The term “continuous analyte sensor” as used herein refers to a device that measures analyte concentration without interruptions for a given time period without requiring user initiation and/or interaction for each measurement. The time period may be short or longer, such as for at least 1 hour, at least 6 hours, at least 12 hours, at least 24 hours, at least 36 hours, at least 48 hours, or longer. The sensor systems may be incorporated into a number of detection devices. For instance, the sensor system may be incorporated into a transdermal analyte monitoring system for discrete and/or continuous glucose monitoring, preferably, continuous glucose monitoring.

The sensor systems described herein contain at least two working terminals (a first working terminal and a second working terminal) and at least one counter terminal. The first working terminal contains an electrode and an enzyme. The first working terminal preferably also contains a hydrogel configured for electrochemical detection of an analyte or analyte indicator, and any non-analyte interferences (also referred to herein as “background interference”).

As used herein, “analyte indicator” means a substance different from the analyte that can be detected and/or measured, and the detection or measurement of the analyte indicator can be correlated (or has a known correlation) to the concentration or presence of the analyte. An exemplary analyte indicator is an analyte reaction product(s). As used herein, “analyte reaction product” refers to a compound formed after physical and/or chemical reactions between the analyte and an enzyme or a non-enzyme component.

For example, an analyte indicator for glucose may be the analyte reaction product, hydrogen peroxide (H₂0₂). In this embodiment, glucose reacts with the enzyme, glucose oxidase, to form gluconic acid and hydrogen peroxide, according to the following reaction:

glucose+0₂ [symbol] gluconolactone+H₂0₂.

Gluconolactone hydrolyses spontaneously to form gluconic acid. The hydrogen peroxide is transported to the surface of the first electrode where it reacts with the surface of the first electrode.

Other analyte indicators include, but are not limited to, binding partners of the analyte for example antibodies, nucleic acids, or complexing agents.

The second working terminal also contains an electrode. Preferably, the second working terminal also contains a hydrogel. However, the second working terminal is configured for electrochemical detection of only background interference, which was also included in the measurement of the first working terminal.

The counter terminal contains a counter electrode for determination of the current produced at the first working terminal and the second working terminal. The sensor system optionally contains more than one counter terminal.

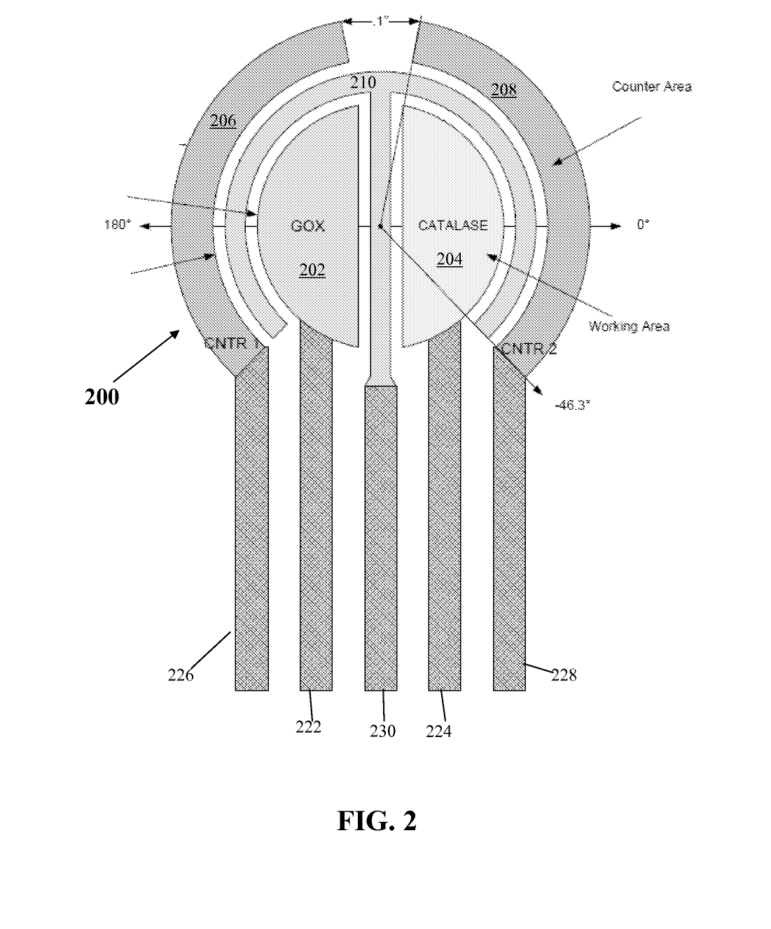

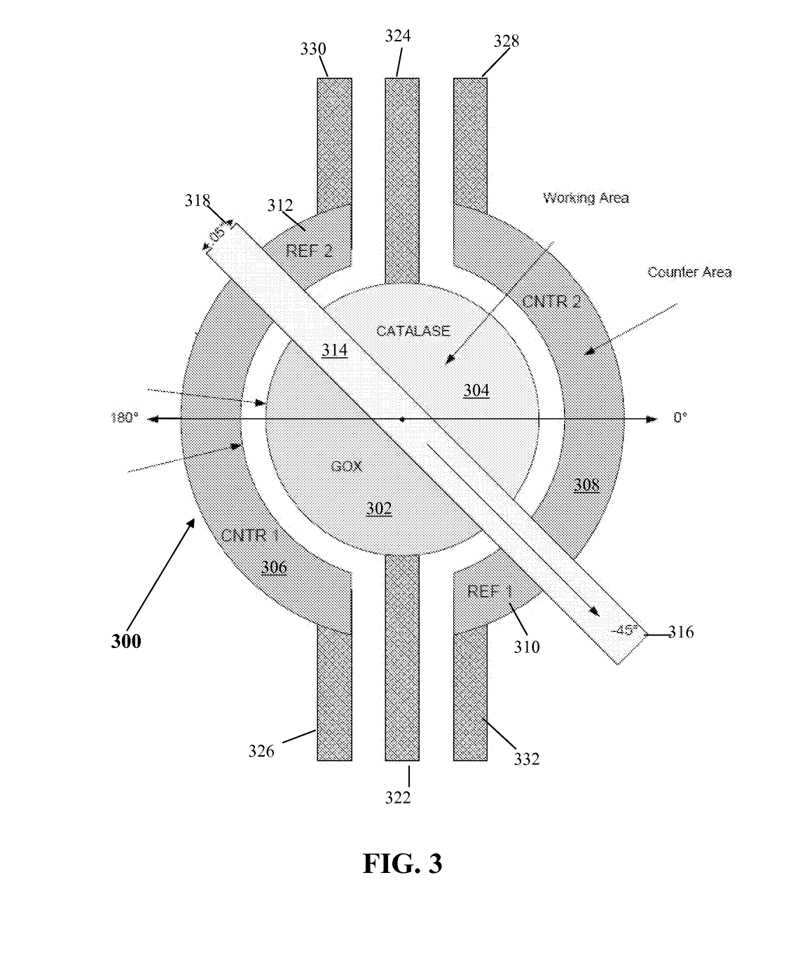

Optionally, the sensor system also contains one or more reference terminals, such as shown in FIGS. 2 and 3. The reference terminal contains a reference electrode, which determines the potential of the electrodes in the working terminals.

### A. First Working Terminal

The first working terminal contains an electrode and an enzyme. Preferably, the first working terminal also contains a hydrogel. The hydrogel contains the enzyme. The electrode and the hydrogel are in electrical communication with each other. The hydrogel may be in the form of a substantially flat body which is positioned directly over the electrode. The hydrogel and the electrode have a suitable shape and size to provide maximum communication there between. The first working terminal may also contain an adhesive ring, which may surround the area in which the electrode and hydrogel are located.

### 1. Measurement of Analyte or Analyte Indicator

The first working terminal uses electrochemical methods to detect and measure the concentration of an analyte that is present in any fluid medium. The first working terminal may be configured to measure the concentration of the analyte or an analyte indicator.

The fluid medium is typically a biological fluid, such as blood, plasma, serum, or interstitial fluid, cerebral spinal fluid, lymph fluid, ocular fluid, saliva, oral fluid, urine, excretions or exudates.

In addition to measuring analyte levels, the first working terminal also detects non-analyte signals when placed on a subject. For example, background interferences due to mechanical, biochemical and/or chemical factors, such as drug interactions, isotonic solution impedances, offset drifts due to thermal response and biosensor and electro-conductive terminal conditioning effects, may also be included in the measurement of the first working terminal.

The first working terminal generates an electrical signal associated with the analyte level and any non-analyte interferences.

### i. Analytes

Any analyte of interest can be measured or detected using the transdermal analyte monitoring system described herein. The term “analyte”, as used herein, refers to a substance or chemical constituents in a biological fluid (for example, blood, interstitial fluid, cerebral spinal fluid, lymph fluid, urine, ocular fluid, saliva, oral fluid, urine, excretions or exudates) that can be analyzed.

Analytes include naturally occurring substances, artificial substances, metabolites, and/or reaction products. Other analytes that can be monitored, include, but are not limited to, glucose, lactate, carbon dioxide, oxygen, electrolytes, ammonia, proteins, albumin, alkaline phosphatase, alanine transaminase, aspartate aminotransferase, bilirubin, blood urea nitrogen, calcium, chloride, creatinine, glucose, gamma-glutamyl transpeptidase, hematocrit, lactate dehydrogenase, magnesium, phosphorus, potassium, sodium, total protein, uric acid, metabolic markers, and drugs. Other analytes that can be detected and measured include, but are not limited to, drugs and metabolites thereof, such as acetaminophen, dopamine, ephedrine, terbutaline, ibuprophen; vitamins, such as Vitamin C (or L-ascorbic acid); uric acid, d-amino acid oxidase, plasma amine oxidase, xanthine oxidase, NADPH oxidase, alcohol oxidase, alcohol dehydrogenase, pyruvate dehydrogenase, diols, reactive oxygen species (ROS), nitric oxide (NO), bilirubin, cholesterol, triglycerides, gentisic acid, L-Dopa, methyl dopa, salicylates, tetracycline, tolazamide, tolbutamide, acarboxyprothrombin; acylcarnitine; adenine phosphoribosyl transferase; adenosine deaminase; albumin; alpha-fetoprotein; histidine/urocanic acid, homocysteine, phenylalanine/tyrosine, tryptophan; andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, human cytomegalovirus (HCMV), HIV-1, human T-lymphotropic virus (HTLV-1), Leber hereditary optic neuropathy, medium-chain acyl-CoA dehydrogenase (MCAD), RNA, phenylketonuria (PKU), Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

The analyte to be measured may be a salt, sugar, protein, fat, vitamin, or hormones naturally occurring in blood or interstitial fluids. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine.

The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, Advanced Glycation End Products (AGEs) and 5-hydroxyindoleacetic acid (FHIAA), or any other biological species.

In a preferred embodiment, the analyte for measurement by the sensor system is glucose.

### 2. Hydrogel

The first working terminal can contain a hydrophilic polymer substrate, such as a hydrogel (also referred herein as “the first hydrogel”), designed to receive the analyte from the skin. The first hydrogel contains an enzyme. The hydrogel provides one or more of the following features: (a) a biocompatible, non-biofouling surface appropriate for long-term exposure to biological fluids without compromise of sensor function, (b) a reservoir for the enzyme, (c) a matrix that can be incorporated with ionic moieties to enhance entrapment of the enzyme, (d) a matrix that can be modulated in terms of its physical and chemical properties (network density, swelling) by varying the molecular weight of the backbone, (e) a transport medium for easy diffusion of the analyte, analyte indicators, and non-analyte components into the electrode, and (e) a matrix that can be rendered bioadhesive by addition of ionic excipients such as chitosan gluconate, polyacrylic acid, poly(amidoamine), poly(ethyleneimine) and hyaluronic acid.

The hydrogel may have any suitable shape that fits in the sensor system. In the exemplary sensor systems shown in FIGS. 1, 2, and 3, the shape of the hydrogel is a substantially flat, semi-circular body. The hydrogel can vary in thickness. Typically the thickness of the hydrogel ranges from about 10 to about 1000 μm, preferably from about 50 to about 700 μm, more preferably from about 200 to about 500 μm.

Suitable polymers that can form the hydrogel include, but are not limited to, synthetic or natural polymers. Examples of suitable synthetic polymers include polyacrylic and polymethacrylic acid polymers, cellulose derivatives such as hydroxypropyl cellulose, polyethyleneglycol polymers, copolymers and block copolymers, and other water swellable, biocompatible polymers. Examples of natural polymers include collagen, hyaluronic acid, gelatin, albumin, polysaccharides, and derivatives thereof. Natural polymers can also be of the type isolated from various plant materials, such as psyllium.

Classes of materials that may be used as the hydrogel include, but are not limited to, PEG-based hydrogels, including polyethylene glycol diacrylate (PEGDA)-based hydrogels, polyethylene glycol diacrylate/polyethyleneimine (PEGDA-PEI) and polyethylene glycol diacrylate-n-vinyl pyrrolidone (PEGDA-NVP), agarose based hydrogels, and vinyl acetate-based hydrogels.

Structurally, polymeric hydrogels are three-dimensional macromolecular configurations. They may be produced through several methods: a) synthesis from monomers (cross-linking polymerization); b) synthesis from polymers and polymerization auxiliary (grafting and cross-linking polymerization); c) synthesis from polymers and non-polymerization auxiliary (cross-linking polymers); d) synthesis from polymers with energy sources (cross-linking polymers without auxiliaries) and e) synthesis from polymers (cross-linking by reactive polymer-polymer intercoupling) (see Hoffman, A., Advanced Drug Delivery Reviews, 2002, 43, 3-12).

### i. PEG-based Hydrogels

The hydrogel utilized in the sensor system is preferably a PEG-based hydrogel, more preferably a PEGDA-based hydrogel. U.S. Pat. No. 8,224,414 to Kellogg et al. (the disclosure of which is incorporated herein by reference) discloses some suitable PEG-based hydrogels in a sensor system. The amount of crosslinker, if one is used to form the hydrogel, is present in an exceedingly small amount and serves to hold the hydrogel together.

Structurally, PEG is highly hydrophilic and presents a high degree of solvation in aqueous solvents. The preferential solvation of PEG molecules can effectively exclude proteins from the PEG chain volume, thereby protecting the surface from bio-fouling by proteins. An advantage that can be provided by chemically crosslinked PEG-based hydrogels is that their physical and chemical properties can be modulated by varying the molecular weight of the PEG chains and varying the initiator concentration. For example, increasing the molecular weight of the polyethylene oxide backbone increases the network mesh size. The release of a bioactive molecule, such as an enzyme, can be controlled by control of the network density. Therefore, a hydrogel containing PEGs of a weight average molecular weight of 8 kDa would have a higher rate of release of an entrapped drug than a hydrogel containing PEGs of a weight average molecular weight of 3.3 KDa.

Optionally, additives can be incorporated into the hydrogels to impart added functionalities, such as bioadhesiveness. For example, hyaluronic acid or polyacrylic acid can be added to the PEG macromer prior to crosslinking to create bioadhesive hydrogels. In another example, an ionic character can be imparted to the crosslinked hydrogels to provide molecular interaction (e.g. ionic bonds) with entrapped drugs, to further control, e.g. slow down rates of release of drug from the matrix.

When the hydrogel is formed from a polyethyleneglycol diacrylate (PEGDA) macromer, polymerization, such as UV polymerization, may occur in a mold. Preferably, the mold contains a pre-loaded support material such as a scrim material, which provides a support matrix for the hydrogel. Preferably, the support material has a suitable configuration to provide a handle, which does not contain the hydrogel. For example, the support material could be in the shape of a half-lollipop, such that the PEGDA macromer polymerizes only around the semi-circular head portion of the material, leaving the tail section of the material hydrogel-free and useful as a handle.

Optionally, the PEGDA based-hydrogel includes an acrylate-PEG-NHS (A-PEG-N) reagent (e.g. sold by Nektar), which can function as a linker molecule to covalently link an enzyme, such as the GOx enzyme, to the PEGDA hydrogel network.

### ii. Agarose-based Hydrogels

An agarose gel may be formed, for example, from 1-20% agarose in buffer solution containing 0-1 M sodium or potassium phosphate, 0-1 M sodium chloride, 0-1 M potassium chloride, 0-2 M lactic acid, surfactant such as 0-1 M TRITON® X-100 (Union Carbide Chemicals & Plastics Technology Corp.), TWEEN® 80 (ICI Americas Inc.) or sodium lauryl sulfate, and any other biocompatible components. Loading of glucose oxidase in agarose hydrogel can be up to 0-20% (by weight), for example, by soaking the solid hydrogel in concentrated glucose oxidase solution, or alternatively by mixing concentrated glucose oxidase powder or solution with agarose solution during its melting stage (15-65° C.), followed by cooling and gelling at lower temperature (40° C. or lower).

### iii. Vinyl Acetate-Based Hydrogels

Alternatively, the hydrogel may be a vinyl acetate-based hydrogel. Vinyl acetate-based hydrogels, include hydrogels formed from the n-vinylpyrrolidone/vinyl acetate copolymer.

### iv. Modified Hydrogels

### a. Covalently Immobilized Hydrogel

Optionally, the hydrogel may be modified to include one or more enzymes and/or humectants. U.S. Publication No. 2008/0281178 to Chuang et al. discloses transdermal analyte monitoring systems that contain a hydrogel with an enzyme and optionally a humectant included therein. The enzymes and/or humectants may be entrapped by any suitable means, including covalent bonding and non-covalent immobilization. Examples of non-covalent immobilization include, but are not limited to ionic interactions and physical entrapment. Preferably the enzymes are covalently linked to the hydrogel, such as by using a linker molecule. In one embodiment, particularly suitable for use in a continuous glucose monitoring system, glucose oxidase (GOx) is covalently immobilized in the hydrogel. For example, covalent immobilization of GOx into a PEGDA network improves the performance of the device by eliminating GOx diffusion (maintaining bioavailability) and/or by stabilizing the enzyme (maintaining bioactivity).

The hydrogel preferably contains greater than 50% (w/w), greater than 60% (w/w), greater than 70% (w/w), greater than 80% (w/w) or greater water. In some embodiments, the hydrogel contains between about 70% to about 80% (w/w) water. The crosslinked polymer acts as an aqueous reservoir to hold components in solution (e.g., buffer salts and osmotic agents), and also provides a transport medium for the diffusion of the analyte.

The PEGDA network provides the structure to contain ˜80% water within its matrix. The enzyme, such as GOx may be physically entrapped or covalently linked to the hydrogel.

### Covalently Linking the Enzyme to the Hydrogel Using a Linker

The coupling of the enzyme to the hydrogel may also be accomplished using a linker. The linker molecule generally contains two or more functional groups which are able to react with functional groups on the enzyme and functional groups on the hydrogel. For example, the linker molecule may contain electrophilic groups which react with nucleophilic groups found in the enzyme and hydrogel, such as hydroxy, thiol, and/or amino groups. These linkers mediate the conjugation of the enzyme to the surface of the hydrogel by forming a bond containing a variable number of atoms. The linker molecules can be homofunctional (i.e., the functional groups are identical) or heterofunctional (i.e., the functional groups are different).

Suitable linker molecules include, but are not limited to, N-Succinimidyl 3-(2-pyridyldithio)propionate (SPDP, 3- and 7-atom spacer), long-chain- SPDP (12-atom spacer), (Succinimidyloxycarbonyl-α-methyl-2-(2-pyridyldithio) toluene) (SMPT, 8-atom spacer), Succinimidyl-4-(N-maleimidomethyl)cyclohexane-1-carboxylate) (SMCC, 11-atom spacer) and Sulfosuccinimidyl-4-(N-maleimidomethyl)cyclohexane-1-carboxylate, (sulfo-SMCC, 11-atom spacer), m-Maleimidobenzoyl-N hydroxysuccinimide ester (MBS, 9-atom spacer), N-(γ-maleimidobutyryloxy)succinimide ester (GMBS, 8-atom spacer), N-(γ-maleimidobutyryloxy) sulfosuccinimide ester (sulfo-GMBS, 8-atom spacer), Succinimidyl 6-((iodoacetyl) amino) hexanoate (SIAX, 9-atom spacer), Succinimidyl 6-(6-(((4-iodoacetyl)amino)hexanoyl)amino)hexanoate (SIAXX, 16-atom spacer), 1,4-Di-[3′-2′-pyridyldithio)propion-amido]butane (DPDPB, 16-atom spacer), Bismaleimidohexane (BMH, 14-atom spacer), and p-nitrophenyl iodoacetate (NPIA, 2-atom spacer). One of ordinarily skill in the art will also recognize that other coupling agents, with different number of atoms, may be used.

Spacer molecules may be incorporated into the linker to increase the distance between the reactive functional groups at the termini, such as acrylate-polyethylene glycol-N-hydroxy succinimide (acrylate-PEG-NHS or A-PEG-N). A number of multifunctional PEGs are commercially available from Shearwater Polymers (Huntsville, Ala.) and Texaco Chemical Co. (Houston, Tex.). Multi-amino PEGs are available under the name “Jeffamine” and include diamino PEGs and triamino PEGs. In the preferred embodiment, the enzyme is covalently immobilized in the hydrogel using an acrylate-PEG-NHS (A-PEG-N).

### Covalently Linking the Enzyme to the Hydrogel Using a Coupling Agent

The enzyme can also be coupled directly to the hydrogel by the use of a reagent or reaction that activates a group on the surface of the hydrogel or the enzyme making it reactive with a functional group on the enzyme or hydrogel, respectively, without the incorporation of a coupling agent.

For example, carbodiimides (CDI) mediate the formation of amide linkages between a carboxylate and an amine or phosphoramidate linkages between phosphate and an amine. Examples of carbodiimides are 1-ethyl-3-(3-dimethylaminopropyl)carbodiimide hydrochloride (EDC), 1-cyclohexyl-3-(2-morpholinoethyl)carbodiimide (CMC), dicyclohexyl carbodiimide (DCC), diisopropyl carbodiimide (DIC), and N,N′-carbonyldiimidazole (CDI). N-ethyl-3-phenylisoxazolium-3′-sulfonate (Woodward's reagent) mediates the formation of amide linkages though the condensation of carboxylates and amines. CDI can also be used to couple amino groups to hydroxyl groups.

### 3. Enzyme

The first hydrogel contains an enzyme (also referred to herein as “the first enzyme”). The enzyme may assist in converting the analyte into a species that can be detected by the electrode. The enzyme acts as a catalyst for a single reaction, converting an analyte into a product that can be detected electrochemically. In a preferred embodiment of a glucose sensor system, the enzyme, glucose oxidase (GOx) is provided to react with glucose (the analyte) and oxygen to form hydrogen peroxide.

Other exemplary enzymes include but are not limited to oxidases, peroxidases, phosphatases, esterases, glycosidases, proteases, hydroxylases, oxygenases, reductases, cellulases, lipases, anhydrase, and amylases, and dehydrogenases. Examples of such enzymes include glucose dehydrogenase, lactate oxidase, alcohol oxidase, pyruvate oxidase, uricase, aldehyde oxidase, xanthine oxidase, choline oxidase, acetylcholine oxidase, glutamate oxidase, glutaminase, glucoamylase, amyloglocosidase, invertase, mutarotase, galactose oxidase, cholesterol oxidase, amino acid oxidase, creatinase, creatininase, sarcosine oxidase, carbonic anhydrase, NAD(P)H oxidase, glycerol-3-phosphate oxidase, thiamine oxidase, pyruvate oxidase, pyridoxal oxidase, D-amino acid oxidase, L-amino acid oxidase, urease, alkaline phosphatase and horseradish peroxidase.

In one embodiment, the first hydrogel may not contain an enzyme. In this embodiment, the first working terminal directly converts the chemical signal associated with the analyte into an electrical signal by chemical reaction at the catalytic face of the working electrode. Thus, the analyte can be detected electrochemically by the first working terminal without first undergoing a reaction catalyzed by an enzyme.

### i. Glucose Oxidase

In the preferred embodiment, the analyte for quantification is glucose, and the enzyme in the first hydrogel is glucose oxidase. Glucose oxidase converts glucose to an analyte indicator that can be converted into an electrical signal by chemical reaction at the catalytic face of the working electrode. Glucose reacts with glucose oxidase to form gluconic acid and hydrogen peroxide, according to the following reaction: glucose+0₂ [symbol] gluconolactone+H₂0₂. Gluconolactone hydrolyses spontaneously to form gluconic acid. The hydrogen peroxide is transported to the surface of the first electrode where it reacts with the surface of the first electrode

Glucose oxidase is preferably present in the hydrogel in an effective amount to continuously react with glucose that contacts the hydrogel over at least 6 hours, 12 hours, 24 hours, 36 hours, or longer.

### 4. Working Electrode

The working electrode of the first working terminal (also referred herein as the first working electrode) is configured to measure the concentration of an analyte. The working electrode and the hydrogel are in communication with each other.

The term “working electrode” refers to an electrode that detects a chemical signal by catalyzing the conversion of a chemical compound into an electrical signal (e.g., conversion of hydrogen peroxide into two electrons, molecular oxygen, and two hydrogen atoms).

The term “chemical signal”, “electrochemical signal”, or “electrochemically active compound” means the chemical compound that is ultimately converted to an electrical signal and measured by the working electrode in conjunction with a monitoring device. Chemical signals can be (1) directly converted into an electrical signal by chemical reaction at the catalytic face of the working electrode, or (2) indirectly converted into an electrical signal by the action of one or more catalysts. For example, where the analyte to be measured in glucose, the chemical signal glucose is indirectly converted into an electrical signal by reactions directed by two catalysts. The first catalyst, the enzyme glucose oxidase, converts glucose into gluconic acid and hydrogen peroxide. Hydrogen peroxide is then converted to an electrical signal by a second catalyst which is the catalytic material (e.g., metal or metal oxide on the catalytic face of the working electrode).

In a preferred embodiment, where the analyte to be detected is glucose, for example, the working electrode measures the hydrogen peroxide produced by an enzyme catalyzed reaction of the analyte being detected and creates a measurable electrical current. Hydrogen peroxide reacts with the surface of the working electrode producing two protons (2H⁺), two electrons (2e⁻) and one molecule of oxygen (O₂). An electric current is generated during this electrochemical oxidation. The amount of current produced is indicative of the rate of hydrogen peroxide produced in the hydrogel, which is related to the amount of glucose flux through the skin (i.e. the rate of glucose flow through a fixed area of the skin). The glucose flux through the skin is proportional to the concentration of glucose in the blood of the user. The working electrode also generates an electrical signal associated with non-analyte background interferences.

An exemplary configuration is illustrated in FIG. 1. The working electrode (not shown) may be patterned over a lead (122) so that it is in electrical contact with the lead (122). The lead can be patterned, using screen printing or other methods known in the art, onto the sensor system so that the sensor system is in electrical communication with external devices or components.

The electrode may be in any suitable shape that fits in the sensor system. In exemplary embodiments, such as FIGS. 1, 2, and 3, the working electrodes are located at the center of the sensor system. Variation of the materials for the first working electrode, coating and dimensions thereof can be made by one of ordinary skill in the art to configure the working electrode to a particular sensor system.

### i. Materials

The working electrode of the first working terminal can be made of any suitable conducting material including, for example, metals and conducting polymers. In one embodiment, the first working electrode contains a catalytic and/or conductive material, such as pure platinum, platinized carbon, glassy carbon, carbon nanotube, mezoporous platinum, platinum black, paladium, gold, or platinum-iridium, for example. In a preferred embodiment, the first electrode contains catalytic cobalt-phthalocyanine carbon.

### ii. Shape

The working electrode can have any suitable shape and size to cover the site for analyte transmission and measure the flux/concentration of the analyte or an analyte indicator. For example, the working terminal may be in the shape of a semi-circle in the center of the sensor system.

The surface area ratio of the first working electrode to the second working electrode is typically about 1:1. In the exemplary embodiments, shown in FIGS. 1, 2, and 3, the surface area ratio of the first working electrode to the second working electrode is 1:1.

The ratio of the combined surface areas for the first and second working electrodes to the surface area of the counter electrode is typically about 1:1 to about 1:5. In one exemplary embodiment, illustrated in FIG. 1, the ratio of the combined surface areas for the first and second working electrodes to the surface area of the counter electrode is 1:1. In another exemplary embodiment illustrated in FIG. 3, the combined surface areas for the first and second working electrodes to the surface area of the counter electrode is 1:0.75.

In exemplary embodiments, such as illustrated in FIGS. 1 and 3, the combined surface area of the first and second working electrodes is approximately 80 mils². In another exemplary embodiment, such as illustrated in FIG. 2, the combined surface area of the first and second working electrodes is approximately 53 mils². The combined surface area of the first and second working electrodes is preferably from about 25 to about 100 mils².

### iii. Spacing

In a typical sensor system, each terminal is separated from the proximal terminal(s) by a sufficient distance, such as from about 20 to about 50 mils². Optionally, the sensor system contains an isolated circuit to prevent the transfer of electrons between the electrodes. In exemplary embodiments, such as illustrated in FIGS. 1, 2, and 3, the electrodes are separated from each other by a distance of approximately 1 mm or greater.

The electrodes are preferably separated from each other by an insulating material to prevent electron transfer from one electrode to another electrode in the same sensor system.

### B. Second Working Terminal

Like the first working terminal, the second working terminal contains an electrode (also referred herein as the “second working electrode”). Preferably, the second working terminal also contains a hydrogel (also referred to herein as the “second hydrogel”). The second working terminal optionally contains an enzyme. The enzyme is typically in the second hydrogel. The second working electrode and the second hydrogel are in electrical communication with each other. The hydrogel may be in the form of a substantially flat body positioned directly over the electrode. The hydrogel and the electrode have a suitable shape and size to provide maximum communication there between. The sensor system may also contain an adhesive ring, which may surround the area in which the electrode and hydrogel are located.

Preferably the first and second working terminals have substantially the same size (e.g. surface area) and shape.

### 1. Measurement of Interferences

The second working terminal is configured to measure background interferences. The electrode in the second working terminal only measures non-analyte signals. The second working terminal measures the same background interferences as measured by the first working terminal, when the first working terminal measures the analyte signal. However, the second working terminal does not detect and/or measure the analyte and/or analyte indicator, particularly analyte reaction product(s).

### i. Interferences

The term “background interferences” as used herein refers without limitation, to any signal that is not related to the analyte or analyte indicator that is measured by the terminal when placed on the subject's skin. Non-analyte signals may be, for example, mechanically, biochemically, and/or chemically derived.

Mechanical factors that produce background interferences may be due to a variety of conditions, including but not limited to, macro-motion of the sensor, micro-motion of the sensor, pressure on the sensor, local tissue stress, and the like.

Interferences caused by biochemical and/or chemical factors may arise from the biological fluid that is being analyzed and include compounds with electroactive acidic, amine or sulfhydryl groups, urea, lactic acid, phosphates, citrates, peroxides, amino acids (e.g., L-arginine), amino acid precursors or break-down products, nitric oxide (NO), NO-donors, NO-precursors or other electroactive species or metabolites produced during cell metabolism and/or wound healing.

Other possible background interferences include, but are not limited to drug interactions, isotonic solution impedances, offset drifts due to thermal response or biosensor and electro-conductive terminal conditioning effects.

Since both working terminals are substantially symmetrical to each other, and due to the sensor's small size, the working terminals are substantially equally affected by mechanical and/or biochemical/chemical factors or other sources of background interference that affect the sensor.

### 2. Hydrogel

Like the first working terminal, the second working terminal contains a hydrogel designed to receive non-analyte interferences from the skin. The second hydrogel optionally contains an enzyme (also referred to herein as the second enzyme). The same hydrogel materials, sizes, and shapes as described above with respect to the first hydrogel may be used for the second hydrogel. In one embodiment, the second hydrogel material is the same as the first hydrogel material; preferably a PEG-based hydrogel. In another embodiment the second hydrogel is formed of a different material than the first hydrogel.

In one embodiment, the first and second hydrogels have substantially the same shape and/or size. In another embodiment the second hydrogel has a different shape and/or size than the first hydrogel.

### 3. Enzyme

The enzyme in the second hydrogel may be an inhibitor of the analyte or one or more of the analyte indicators, preferably one or more analyte reaction products, to prevent electrochemical detection of the analyte, or its reaction products, by the second electrode. In a preferred embodiment, particularly when the analyte is glucose, the enzyme in the second hydrogel is catalase. Other suitable enzymes include, but are not limited to, peroxidases, oxidases, phosphatases, esterases, glycosidases, proteases, cellulases, lipases, anhydrase, and amylases.

### i. Catalase

In the preferred embodiment, such as when the analyte to be measured is glucose, the enzyme in the second hydrogel is catalase. Catalase breaks down hydrogen peroxide to water and molecular oxygen and prevents electrochemical detection by the second electrode.

### 4. Second Working Electrode

The second working electrode is configured to measure background interferences, which are the same as the interferences measured by the first working electrode. The second working electrode and the hydrogel are in electrical communication with each other. Any change in the electrical signal detected by the second working electrode correlates with changes due to background interferences.

As shown in FIG. 1, in one embodiment, the working electrode (not shown) may be patterned over a lead (124) so that it is in electrical contact with the lead (124). The lead can be patterned, using screen printing or other methods known in the art, onto the sensor system in a manner that permits electrical communication to external devices or components.

In exemplary embodiments, such as FIGS. 1, 2, and 3, the working electrodes are located at the center of the sensor system. The second working electrode may be in any suitable shape that fits in the sensor system. Variation of the materials for the first working electrode, coating and dimensions thereof can be made by one of ordinary skill in the art to configure the working electrode to a particular sensor system. In a preferred embodiment, the second working electrode has similar shape and dimensions as the first working electrode.

### i. Materials

Like the first working electrode, the second working electrode can be made of any suitable conducting material including, such as for example, metals and conducting polymers.

In one embodiment, the second working electrode is an active catalytic cobalt-phthalocyanine carbon electrode. When the electrode is active, the hydrogel of the second working terminal contains a second enzyme to prevent electrochemical detection of the analyte or analyte reaction product.

In another embodiment, the second electrode is a blank carbon electrode. The blank carbon electrode is not active and does not require the presence of a second enzyme. Using a blank carbon electrode as the second working electrode achieves the same effects, i.e. measurement of background interferences, as having an active electrode with a second enzyme, such as catalase.

### ii. Shape

The second working electrode can have any suitable shape and size to cover the site for analyte transmission and measure the electrical signal due to background interferences thereof The working terminal is for example, in the form of a semi-circle in the center of the sensor system.

In a preferred embodiment, the second working electrode has a similar shape and size as the first working electrode.

### iii. Spacing

In a typical sensor system, the second working terminal contains a sufficient space between it and the first working terminal, or any other terminal the second working terminal.

### C. Counter Terminal

In addition to the two working terminals, the sensor system typically contains one or more counter terminals. As is generally known in the art, the inclusion of the counter electrode allows for reduction in the reference electrode's surface area, and thereby allows for further miniaturization of the sensor (e.g. reduction in the sensor's size and/or surface area, etc.).

The counter electrode serves to make an electrical connection to the electrolyte so that a current can be applied to the working electrode(s).

The counter terminal contains one or more electrodes (also referred to herein as the “third electrode” or “counter electrode”). In one exemplary embodiment, such as illustrated in FIG. 1, the sensor system (100) contains one counter electrode (106), which is located about the periphery of the two working electrodes. In other exemplary embodiments, such as illustrated in FIGS. 2 and 3, the sensor system contains two counter electrodes (206 and 208). The counter electrodes may be located about the periphery of the reference terminal (210) (FIG. 2), or about the periphery of the working electrodes (FIG. 3).

### 1. Measurement of Current from Working Terminals

The counter terminal(s) is/are provided to determine the current produced at the first working electrode and the second working electrode. For example, in one embodiment, the current produced at the first working electrode from hydrogen peroxide oxidation and background interferences flows through the circuitry to the counter electrode and is substantially proportional to the amount of hydrogen peroxide that diffuses to the first working electrode and background interferences. Similarly, the current that is produced at the second working electrode from background interferences flows through the circuitry to the counter electrode and is substantially proportional to the amount of background interference that the second working electrode measures.

### 2. Counter Electrode

The counter electrode(s) is/are operatively associated with the first electrode and the second electrode. The counter electrode (not shown) may be patterned over lead (126) so that it is in electrical contact with the lead (126). The lead can be patterned, using screen printing or other methods known in the art, onto the sensor system in a manner that permits electrical or wireless communication with external devices or components.

The counter electrode may be in any suitable shape that fits in the sensor system. Variation of the materials for the counter electrode, coating and dimensions thereof can be assessed.

### i. Materials

Like the working electrodes, the counter electrode(s) can be made of any suitable conducting material including, for example, metals and conducting polymers. The counter electrode is usually made of inert materials (noble metals or carbon/graphite) to avoid its dissolution. In a preferred embodiment, the counter electrode contains silver and silver chloride.

### ii. Shape

The counter terminal(s) can have any suitable shape and size. The counter terminal may have a suitable size and shape to surround the outer portion of the perimeter of all or a portion of the working terminals, such as illustrated in FIGS. 1 and 3.

The ratio of the combined surface areas of the working terminals to the surface area of the counter terminal(s) ranges from about 1:1 to about 1:5. In the embodiment illustrated in FIG. 1, the ratio of the combined surface areas of the working terminals to the surface area of the counter terminal is 1:1. In the embodiment illustrated in FIG. 3, the ratio of the combined surface areas of the working terminals to the surface area of the counter terminal is 1:0.75.

Typical surface areas for the counter terminal (or the combined surface area for more than one counter terminal) range from 25 to 500 mils². For example, as illustrated in FIG. 1, the surface area of the counter electrode is approximately 160 mils². In another exemplary embodiment, such as illustrated in FIG. 2, the combined surface area of the first counter electrode and the second counter electrode is approximately 106 mils². In another exemplary embodiment, such as illustrated in FIG. 3, the surface area of the combined first counter electrode and second counter electrode is approximately 123 mils².

### D. Reference Terminal

The sensor system optionally includes one or more reference terminals. The reference terminal(s) is/are operatively associated with the first working terminal and the second working terminal. The reference terminal measures the potential of the working electrodes.

The reference terminal contains an electrode (also referred to herein as the “fourth electrode” or “reference electrode”). The reference electrode may be patterned over a lead so that it is in electrical contact with the lead. The lead can be patterned, using screen printing or other methods known in the art, onto the sensor system in a manner that permits electrical or wireless communication with external devices or components.

### 1. Reference Electrode

The reference electrode(s) is/are operatively associated with the first electrode and the second electrode. The reference electrode may be in any suitable shape that fits in the sensor system.

The reference electrode has a stable and well-known electrode potential. The high stability of the electrode potential is usually reached by employing a redox system with constant (buffered or saturated) concentrations of each participants of the redox reaction.

Basically, the reference electrode serves as a half cell to build an electrochemical cell. This allows the potential of the other half cell (i.e. the working electrodes) to be determined.

The reference electrode(s) can be made of any suitable conducting material that provides a stable, well-defined electrode potential. Suitable materials include, for example, metals and conducting polymers. Common reference electrodes contain binary oxi-reductive materials that provide consistent redox potential. Suitable reference electrodes include but not limited to the saturated calomel electrode (SCE), Ag/AgCl, and Cu/CuSO₄ electrodes. In a preferred embodiment, the reference electrode preferably contains silver and silver chloride.

The reference electrode may have any suitable shape that fits in the sensor system. Preferably the reference terminal is located about the outer perimeter of the working terminal, and optionally also located between the working terminals. In one exemplary embodiment, such as illustrated in FIG. 2, the reference terminal (210) is located about the periphery and between the working terminals. In another exemplary embodiment, such as illustrated in FIG. 3, the sensor system (300) comprises a first reference terminal (310) and a second reference terminal (312). The first reference terminal (310) and the second reference terminal (312) are positioned about the periphery of the first working terminal and the second working terminal, respectively.

Variation of the materials for the reference electrode, coating and dimensions thereof can be determined by one skilled in the art.

### E. Exemplary Sensor Systems

Some exemplary configurations of the sensor system are illustrated in FIGS. 1, 2 and 3 and described herein. Those skilled in the art will appreciate that the configurations can be modified, yet still achieve the desired result, i.e. reduction, or elimination, of background interferences. For example, the location of the electrodes can be alternated.

### 1. Three Terminal Sensor System

FIG. 1 is a schematic of an exemplary embodiment of a three-terminal sensor system. The sensor system (100) includes a first working terminal (102), a second working terminal (104), and a counter terminal (106). The working terminals are located in the center of the sensor system. The first working terminal is configured to generate an electrical signal associated with an analyte or an analyte indicator. The second working terminal is configured to generate an electrical signal associated with background interferences.

The counter terminal (106) is generally positioned about the periphery of at least a portion of each of the working terminals (102 and 104). The counter electrode is operably associated with the first working electrode and the second working electrode.

The sensor system also contains leads (122, 124, and 126) which are in electrical communication with the electrodes and are connected to or in electrical communication with an external device or other components that provide a user interface and/or print-out relating the analyte level to the user.

### 2. Five Terminal Sensor System

FIG. 2 is a schematic of an exemplary embodiment of a five terminal sensor system. The sensor system (200) includes a first working terminal (202), a second working terminal (204), a first counter electrode (206), a second counter electrode (208), and a reference electrode (210). The working terminals are located in the center of the sensor system.

The reference electrode (210) is positioned about the periphery of, and between the first working electrode and the second working electrode (202 and 204). The reference electrode is operably associated with the first working electrode and the second working electrode.

The first counter electrode (206) is operably associated with the first working electrode and may be positioned about a portion of the periphery of the reference electrode (210). The second counter electrode (208) is operably associated with the second working terminal and may be positioned about a second portion of the periphery of the reference electrode (210).

The sensor system also contains leads (222, 224, 226, 228, and 230) which are in electrical communication with the electrodes and are connected to or in communication with an external device or other components configured to provide a user interface or print-out relating the analyte level to the user.

### 3. Six Terminal Sensor System

FIG. 3 is a schematic of an exemplary embodiment of a six terminal sensor system. The sensor system includes a first working terminal (302), a second working terminal (304), a first counter electrode (306), a second counter electrode (308), a first reference electrode (310), and a second reference electrode (312). As shown in FIG. 3, an insulator portion (314) separates the first working electrode from the second working electrode. Additionally, a portion (316) of the insulator portion also separates the first reference terminal (310) from the second counter terminal (308). A second portion (318) of the insulator portion separates the second reference terminal (312) from the first control terminal (306). Alternatively the insulator portion can be provided as separate materials, such as a first insulator, a second insulator and a third insulator, to separate two terminals from each other.

The working terminals are located at the center of the sensor system. The first reference electrode (310) is operably associated with the first working electrode. The second reference electrode (312) is operably associated with the second working electrode. The reference electrodes (310 and 312) and the counter electrodes (306 and 308) may, in combination, be positioned about the periphery of the working electrodes (302 and 304).

The sensor system contains leads (322, 324, 326, 328, 330, and 332) which are in electrical communication with the electrodes at one end. The leads connect to or are in communication with an external device or other components configured to provide a user interface or print-out relating the analyte level to the user.

### II. Transdermal Analyte Monitoring System

Generally, the transdermal analyte monitoring system (“TAMS”) contains a sensor system (as describe above) in electrical or wireless communication with a user interface, display and/or computing device. Suitable means of communication include a wireless connection or any means for an electrical connection, such as a flexible connecting cable. For embodiments in which a reaction between the analyte and an enzyme occurs in the first working terminal, transdermal analyte monitoring system optionally contains at least one opening (such as a channel or pocket) for providing a sufficient amount of oxygen to the hydrogel, particularly the hydrogel in the first working terminal, to allow for the reaction between the enzyme and analyte to occur.

The TAMS is preferably used as a continuous analyte sensor that measures the concentration of an analyte of interest or analyte indicator in a body fluid (e.g. blood, serum, plasma, interstitial fluid, cerebral spinal fluid, lymph fluid, ocular fluid, saliva, or oral fluid).

The TAMS is configured to be applied to an area on the skin of an animal; typically the animal is a mammal, and in the preferred embodiment the mammal is a human. Generally, as the analyte transfers out of the patient's body into the TAMS, the enzyme in the first hydrogel reacts continuously with the analyte, and produces an analyte indicator. In one embodiment, the first hydrogel may not contain an enzyme. In this embodiment, the analyte can be detected electrochemically by the first working terminal without first undergoing a reaction catalyzed by an enzyme.

The chemical signal associated with the analyte or analyte indicator is converted into an electrical signal by chemical reaction at the catalytic face of the working electrode, which correlates with an analyte value and background interferences. Simultaneously with the detection by the first working electrode, the second working electrode detects signals due solely to background interferences, i.e. non-analyte signals that are also detected by the first electrode.

The first working electrode measures the analyte or analyte indicator, reacts with the analyte or analyte indicator, and creates a measurable electrical current. The amount of current produced is indicative of the rate of analyte indicator produced in the hydrogel, which is related to the amount of analyte flux through the skin (e.g., if the analyte is glucose, this correlates with the rate of glucose flow through a fixed area of the skin). The analyte flux through the skin is proportional to the concentration of analyte in the blood of the user. The working electrode also generates an electrical signal associated with non-analyte background interferences.

### A. Sensor System

The sensor system shown in FIGS. 1, 2, and 3 may be incorporated into any one of a number of analyte detection devices. In a preferred embodiment, this sensor system may be incorporated into a transdermal analyte monitoring system (TAMS) to provide discrete or continuous glucose measurement.

### 1. Electrical Signals

The sensor system generates at least two electrical signals, a first electrical signal and a second electrical signal. The first electrical signal correlates with the analyte level and any non-analyte interference detected in real time. The second electrical signal correlates directly with only the non-analyte interferences.

### i. Electrical Signal from the First Working Electrode

The first working electrode produces a first electrical signal due to analyte and background interferences. In the preferred embodiment, where the analyte is glucose, the first working electrode measures the current generated from electrochemical oxidation of hydrogen peroxide, produced from reaction of glucose with the first enzyme. The first working terminal also detects an electrical signal due to non-analyte or background interferences.

### ii. Electrical Signal from the Second Working Electrode

The second working electrode produces a second electrical signal associated with background interferences. Both working electrodes are constructed substantially symmetrically and identically, and are located within the sensor system as physically close to each other as possible. Consequently, the interferences encountered and measured by the first working electrode are substantially the same as the interferences encountered and measured by the second working electrode.

### B. Computing Device

As generally used herein, “computing device” refers to hardware, software, firmware, or combinations thereof that perform a function or an action, and/or cause a function or an action from another component. For example, depending on the application or needs of the system or device, computing device may include a software controlled microprocessor, discrete logic such as an application specific integrated circuit (ASIC), a programmed computing device, memory device containing instructions, or the like. Computing device may also be fully embodied as software configured to perform the desired action or function.

As generally used herein “software” refers to one or more computer readable and/or executable instructions that cause a computer or other electronic device to perform functions, actions, and/or behave in a desired manner. The instructions may be embodied in various forms such as routines, algorithms, modules, or programs including separated applications or code from dynamically linked libraries. Software may also be implemented in various forms such as a stand-alone program, a function call, a servlet, an applet, instructions stored in a memory, part of an operating system or other type of executable instructions. As is appreciated by one of ordinary skill in the art, the form of software is dependent on, for example, requirements of a desired application, the environment it runs on, and/or the desires of a designer/programmer or the like.

The sensor system of the TAMS may be attached by any suitable means to a display or computing device. Suitable means include a wireless connection or any other means for electrical connection, such as a flexible connecting cable.

The computing device comprises systems necessary to receive, process, and display sensor data from the analyte sensor. Particularly, the computing device may be a pager-sized device, for example, and house a user interface that has a plurality of buttons and/or keypad and a liquid crystal display (LCD) screen. However, the computing device may comprise any reasonable configuration, including a desktop computer, laptop computer, a personal digital assistant (PDA), a server (local or remote to the receiver), or the like. In some embodiments, a computing device may be adapted to connect (via wired or wireless connection) to a desktop computer, laptop computer, a PDA, a server (local or remote to the computing device), or the like in order to download data from the computing device.

In some alternative embodiments, the computing device may be housed within or directly connected to the sensor in a manner that allows the sensor and the computing electronics to work directly together and/or share data processing resources. Accordingly, the computing device, including its electronics, may be generally described as a “computer system.” In some embodiments the user interface may also include a speaker, and a vibrator.

The computing device performs mathematical analysis using an appropriate algorithm or signal processing on the electrical information provided by the electrodes (the first electrode and the second electrode) and calculates an analyte value. The analyte value is typically is the flux of the analyte through the subject's skin, the concentration of the analyte in the subject's body fluid, or both.

The computing device is configured to remove the second electrochemical signal, due to non-analyte interferences, from the first electrochemical signal, due to both analyte and non-analyte interferences. In one embodiment, the computing device is configured to subtract the second signal from the first signal. In another embodiment, the subtraction step may be performed digitally in the sensor system. In another embodiment, subtraction may be performed digitally including at least one hardware element and software configured to digitally subtract the second signal from the first signal.

### C. Kits

Kits for measuring an analyte contain the transdermal analyte monitoring system described above and instructions that explain to a user how to use the TAMS. The kit contains one or more sensor systems. Preferably the kit contains more than one sensor system. Preferably each sensor system is designed to be disposed of following a single use.

Optionally, the kit also contains a cleaning system for cleaning the skin prior to application of the TAMS on the skin. In one embodiment, the kit contains one or more wipes, preferably pre-moistened wipes.

Optionally, the kit contains a skin permeabilization device, preferably a controlled skin abrasion device.

### III. Method for Improving Analyte Detection

### A. Skin Permeabilization

Prior to applying the transdermal analyte monitoring system to a site on the patient's skin, the permeability of the skin site is increased. Preferably the stratum corneum is removed in a controlled manner. Any suitable permeabilization device and method may be used to increase the permeability of the skin site. Typical methods for increasing the skin's permeability include abrasion, tape stripping, rubbing, sanding, laser ablation, radio frequency (RF) ablation, chemicals, sonophoresis, iontophoresis, electroporation, application of permeation enhancing agents. Preferably, permeability of the skin is increased to the desired level using a controlled skin abrasion device. Suitable controlled abrasion devices are described in U.S. Publication No. 2008/0275468 to Chuang, et al., the disclosure of which is incorporated herein. Preferably the controlled abrasion device is the Prelude® SkinPrep System (Echo Therapeutics, Inc.).

The permeabilization step is preferably continued until the desired permeability level is achieved, which can be determined by measuring its transepidermal water loss (TEWL). The TEWL can be determined using technologies from cyberDERM Inc. or Delfin Technologies (such as the Vapometer). Preferably, following the permeabilization step, the skin site has a TEWL of between about 20 to 50 g/m²/hr, preferably between about 30 to 40 g/m²/hr. Preferably the skin site is uniformly abraded by a controlled abrasion device to the desired permeability level.

### B. Coupling of the Monitoring System to the Permeabilized Site

The transdermal analyte monitoring system is configured to continuously and accurately measure an analyte level over a time period of at least 6 hours, at least 12 hours, preferably at least 24 hours, more preferably at least 36 hours, most preferably at least 48 hours.

Preferably, one or more attachment means are used to secure the TAMS to the abraded skin site. A variety of attachment means may be used, including but not limited to adhesive, straps, and elastic bands/chords.

### C. Transfer of Analyte to the TAMS

Bodily fluid containing the analyte to be measured may transfer from the patient's body and into the TAMS by any suitable means. However, in the preferred embodiment, the bodily fluid transfers by passive diffusion out of the patient's body and into the TAMS. Alternatively, force can be applied to remove the analyte such as physical forces, chemical forces, biological forces, vacuum pressure, electrical forces, osmotic forces, electromagnetic forces, ultrasound forces, cavitation forces, mechanical forces, thermal forces, capillary forces, fluid circulation across the skin, electro-acoustic forces, magnetic forces, magneto-hydrodynamic forces, acoustic forces, convective dispersion, photo acoustic forces, by rinsing body fluid off skin, and any combination thereof.

In one embodiment, the TAMS is placed over the skin site that has been treated by an abrasion system. The analyte transfers across the skin and into the TAMS. The sensor in the TAMS receives an analyte flux through the skin and provides continuous measurement of the analyte level. Preferably, the analyte to be measured is glucose.

D. Analysis of electrical signals

The first electrical signal and the second electrical signal generated by the sensor system of the TAMS are detected by the computing device. The computing device removes, such as by subtraction, the second signal from the first signal and determines a differential signal which corresponds with at least one analyte level data point. The subtraction can be performed electronically or digitally in the computing device.

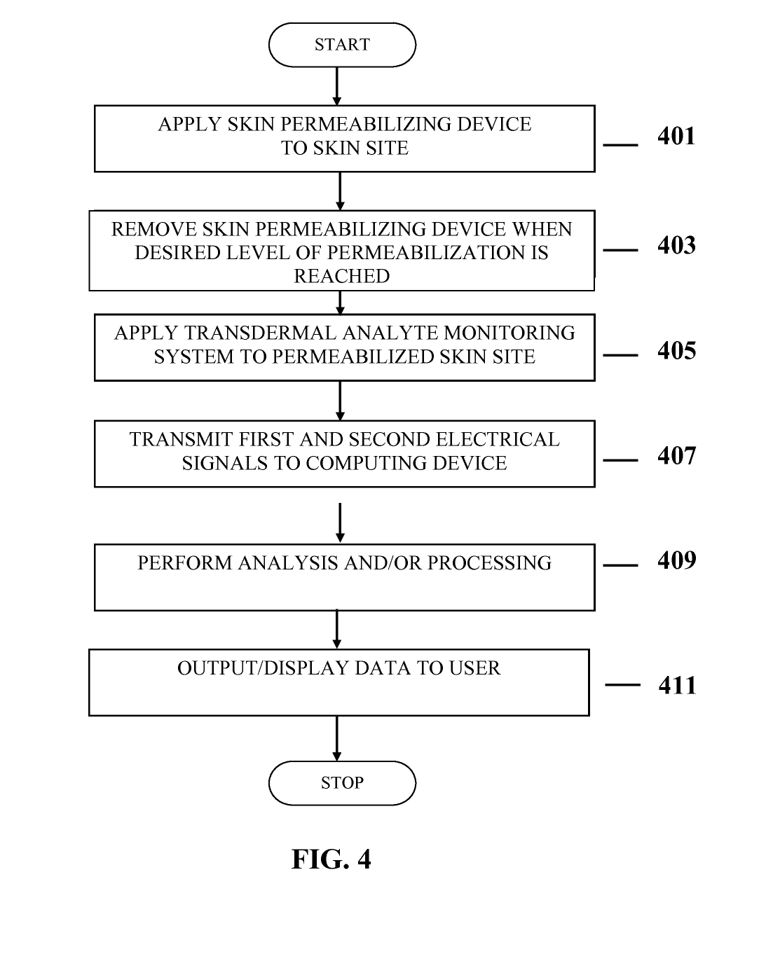

FIG. 4 is a flowchart of an exemplary method for measuring the level of an analyte in a patient. In step 401, the skin is pretreated to enhance permeability of the skin site. Preferably, a controlled abrasion device is applied for a suitable period of time to remove stratum corneum. Next, in step 403, the skin permeabilizing device is removed once the desired level of permeability is achieved. The level of permeability can be determined by any suitable measurement, such as described in U.S. Pat. No. 8,386,027 to Chuang, et al.

In step 405, the TAMS is applied to the permeabilized skin site and a bodily fluid, e.g. interstitial fluid, containing the analyte, is transferred from the patient's body, into the TAMS, such that it contacts both the first and second hydrogels. The TAMS produces at least two electrical signals from the first and second electrodes. For example, in one embodiment, the flux of hydrogen peroxide and background interferences are detected by the first electrode, which produces a first electrical signal. Meanwhile, the second hydrogel blocks hydrogen peroxide from being passed to the second electrode. The second electrode only detects background interferences and produces a second electrical signal.

In step 407, the electrical signal is transmitted to a computing device, which may be remote from the sensor, alternatively the computing device may be included in the sensor.

In step 409, the computing device analyzes and/or process the data. For example, the computing device may subtract the second electrical signal from first electrical signal to determine an analyte level. If necessary, the data is converted to the relevant units, such as glucose concentration measurements. In step 411, parameters describing the analyte level (or concentration) and/or change in the analyte level over time are provided to the user. For example, the output can include analyte indicator level, analyte level, analyte flux, analyte concentration, first and second sensor signals over time. Preferably the data is provided continuously and in real time to the user. The data relating to the analyte level is transmitted to one or more interfaces or a display for viewing by, for example, a patient or a medical professional. After a suitable period of time, the glucose sensor is removed and a new sensor may be placed on the site to monitor the patient's glucose level. Preferably the sensor remains on the patient's skin site for at least 6 hours, at least 12 hours, at least 24 hours, at least 36 hours, at least 48 hours, or longer.

### EXAMPLES

### Example 1

### Studies with Transdermal Glucose Monitoring Sensor

In a 24 hour study, four sensor tests with two glucose oxidase and two non-glucose oxidase biosensors on one human subject were performed. The subject's skin was abraded (site 1 and site 2) using the Abrasion Study Procedure Abrasion Level B1 protocol. Uniform abrasions were made with an approximate TEWL measurement of 30 g/m²/hr. A glucose oxidase (GOX) and non-glucose oxidase (non-GOX) sensor pair was placed on each arm of the subject, as physically close together as the mechanical enclosures allowed.

The sensor data was reviewed both individually and with the GOX data subtracted from the non-GOX for each arm. Utilizing the peak data of current and glucose readings, the slope (gain) of the sensor at the beginning of the test and at the end of the test were calculated to determine if there was a gain shift.

### Site 1—No Glucose Oxidase

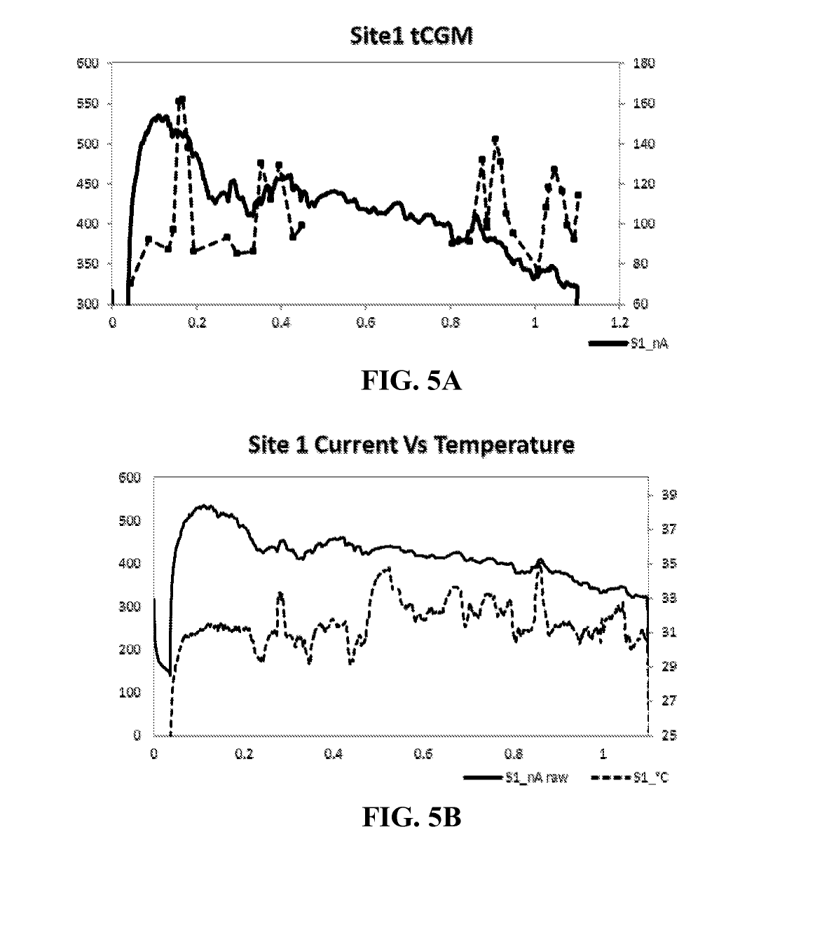

FIGS. 5A and 5B are graphs of the background interferences over time. In FIG. 5A, the black line shows the change in current and the red line shows the change in glucose levels in mg/dL over time at an abraded skin site, site 1. In FIG. 5B, the green line shows the change in current over time and the red line shows the change in temperature (° C.) over time.

### Site 2—With Glucose Oxidase

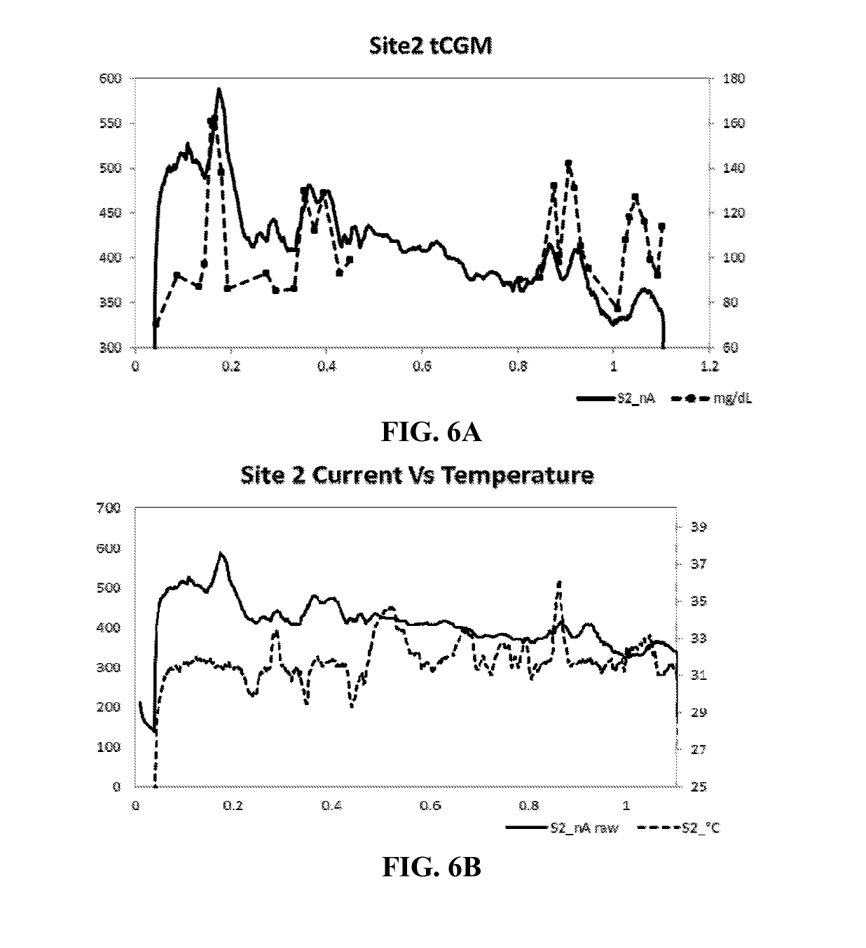

FIGS. 6A and 6B are graphs of the glucose level (mg/dL) over time. In Figure A, the grey line shows the change in current over time and the red line shows the change in glucose levels over time measured in mg/dL at an abraded skin site, site 2. In FIG. 6B, the green line shows the change in current over time and the red line shows the change in temperature (° C.) over time.

### Difference Between the Signals Obtained From Site 2 and Site 1

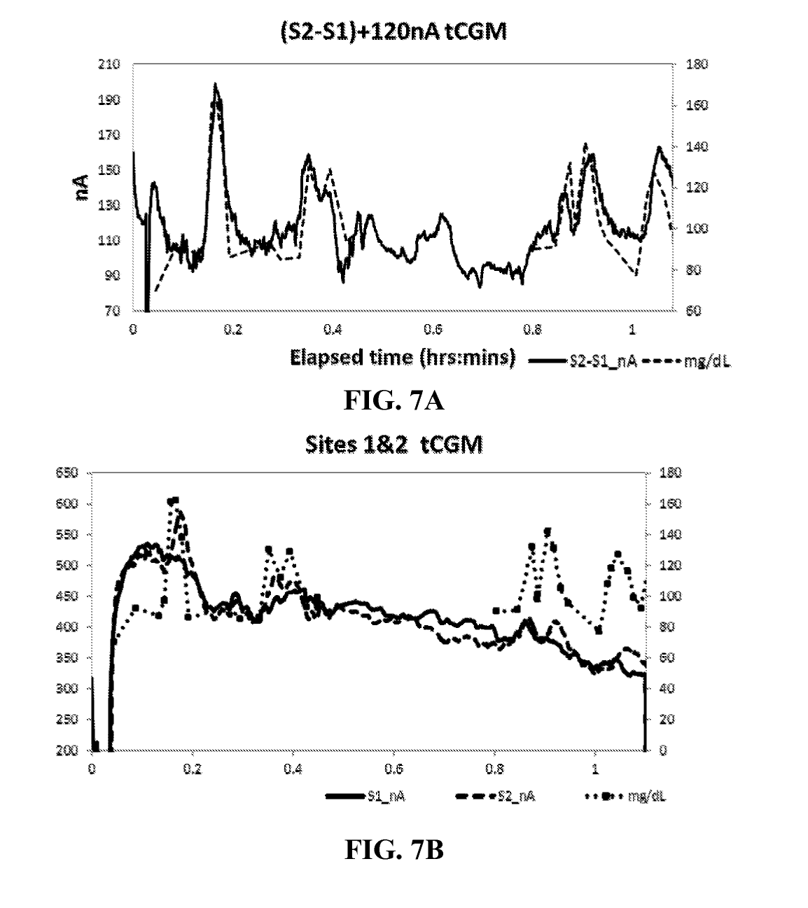

FIG. 7A and 7B are graphs of the difference between the signals obtained from site 1 and site 2. In FIG. 7A, the blue line shows the difference in the background signal and analyte signal over time and the red line shows the change in glucose levels in mg/dL over time. In FIG. 7B, the black line is the background signal, the grey line is the glucose signal, and the red line is the glucose level in mg/dL.

As shown by FIGS. 7A and 7B, the non-glucose oxidase sensor was successful at measuring the on-skin offset and drift effects (i.e. the background interferences). The on-skin offset and drift effects were successfully subtracted from the GOX sensor data yielding a less obstructed, more pure glucose data signature.

We claim:

1. An analyte sensor system comprising:
a first working terminal comprising a first electrode and a first enzyme;
a second working terminal comprising a second electrode and optionally a second enzyme which is different from the first enzyme; and
a counter terminal comprising a third electrode, wherein the counter terminal is configured to receive an electrical signal from each of the working terminals when the sensor system is placed on a site on the skin of a subject.

2. The sensor system of claim 1, wherein the first enzyme reacts with an analyte to produce an analyte reaction product that can be measured by the first electrode.

3. The sensor system of claim 2, wherein the second enzyme inhibits the analyte or the analyte reaction product to prevent electrochemical detection of the analyte or the analyte reaction product by the second electrode.

4. The sensor system of claim 1, wherein
the first electrode comprises an electrically conductive material, preferably carbon and cobalt-phthalocyanine;
the second electrode comprises carbon; and
the third electrode comprises an electrically conductive material, preferably silver and silver chloride.

5. The sensor system of claim 1, wherein the ratio of the surface area for the third electrode to the combined surface area for the first and second electrodes is about 0.75:1 to 5:1, preferably at least 1:1.

6. The sensor system of claim 1, wherein the surface areas of the first and second electrodes are substantially the same.

7. The sensor system of claim 1, wherein
the first working terminal further comprises a first hydrogel comprising the first enzyme, and the first hydrogel is in electrical communication with the first electrode, and
the second working terminal further comprises a second hydrogel comprising the second enzyme, and the second hydrogel is in electrical communication with the second electrode.

8. The sensor system of claim 7, wherein each of the first and second hydrogels are formed from a polymer selected from the group consisting of polyethylene glycol diacrylate (PEGDA), agarose, polyethylene glycol diacrylate/polyethyleneimine (PEGDA-PEI), polyethylene glycol diacrylate-n-vinyl pyrrolidone (PEGDA-NVP), acrylate-polyethylene glycol-N-hydroxy succinimide (A-PEG-N), and blends and copolymers thereof

9. The sensor system of claim 8, wherein the first hydrogel and the second hydrogel are polyethylene glycol diacrylate (PEGDA)-based hydrogels.

10. The sensor system of any one of claims 1 to 9, wherein the enzyme is selected from the group consisting of glucose oxidase, glucose dehydrogenase, lactate oxidase, alcohol oxidase, pyruvate oxidase, uricase, aldehyde oxidase, xanthine oxidase, choline oxidase, acetylcholine oxidase, glutamate oxidase, galactose oxidase, cholesterol oxidase, amino acid oxidase, creatinase, creatininase, sarcosine oxidase, carbonic anhydrase, NAD(P)H oxidase, glycerol-3-phosphate oxidase, thiamine oxidase, pyruvate oxidase, pyridoxal oxidase, D-amino acid oxidase, L-amino acid oxidase, alkaline phosphatase, catalase.

11. The sensor system of claim 10, wherein the first enzyme is glucose oxidase.

12. The sensor system of claim 11, wherein the second enzyme is catalase.

13. The sensor system of any one of claims 1 to 12, further comprising a reference terminal comprising a fourth electrode, wherein the fourth electrode is configured to continuously measure the potential of the working electrodes when the sensor system is placed on a site on the skin of a subject.

14. The sensor system of claim 13, comprising two counter electrodes, two working electrodes, and one reference electrode.

15. The sensor system of claim 13, comprising two counter electrodes, two working electrodes, and two reference electrodes.

16. A transdermal analyte monitoring system comprising:
the sensor system of any one of claims 1 to 15; and
a computing device configured to analyze the electrical signals and provide an output correlating to the analyte level in real time.

17. The transdermal analyte monitoring system of claim 16, wherein in use the system:
a) measures a first electrical signal due to the analyte level and non-analyte interferences, and simultaneously
b) measures a second electrical signal due to non-analyte interferences, and wherein the non-analyte interferences correlate with interference from a subject; and subsequently
c) removes the second electrical signal from the first electrical signal in real time to provide an analyte signal.

18. The transdermal analyte monitoring system of any one of claims 16 to 17, wherein the system comprises at least one opening for providing oxygen to the first hydrogel.

19. A method for improving detection of an analyte level in a subject in need of monitoring comprising:
(a) applying the transdermal analyte monitoring system of any one of claims 16 to 18 to a site on the skin of a subject.

20. The method of claim 19, further comprising prior to step (a), permeabilizing the skin to remove the stratum corneum, preferably wherein the resulting transepidermal water loss (TEWL) of the skin site is between about 20 to 50 g/m²/hr, preferably between about 30 to 40 g/m²/hr.

21. The method of claim 19 or 20, wherein the transdermal analyte monitoring system is continuously applied to the skin for at least 12 hours, at least 24 hours, at least 26 hours, preferably at least 48 hours.

22. The method of claim 21, further comprising removing the transdermal analyte monitoring system after at least 12 hours, at least 24 hours, at least 36 hours, preferably at least 48 hours.
